(19) United States
(12) Patent Application Publication
(10) Pub. No.: US 2022/0290908 A1
(43) Pub. Date: Sep. 15, 2022

(54) IMPROVEMENTS IN OR RELATING TO TEMPERATURE CONTROL PACKAGES

(71) Applicant: SOFTBOX SYSTEMS LIMITED, Long Crendon, Buckinghamshire (GB)

(72) Inventors: Edwin Francis Tattam, North Cheriton (GB); Padraic Thomas O'Hara, Long Crendon (GB); Baptiste Kuhn, Long Crendon (GB)

(21) Appl. No.: 17/633,500

(22) PCT Filed: Aug. 5, 2020
(86) PCT No.: PCT/EP20/25358

§ 371 Date: Feb. 7, 2022

(30) Foreign Application Priority Data
Aug. 5, 2019 (GB) 1911196.2

(51) Int. Cl. F25D 3/08; B65D 81/18; B65D 81/38; B65D 65/46

(52) U.S. Cl. CPC F25D 3/08; B65D 81/18; B65D 81/3823; B65D 65/466; F25D 2303/0843; F25D 2303/0844; F25D 2303/0845; F25D 2303/08222

(57) ABSTRACT

The present invention relates to temperature control packages, such as coolant packages that are used to maintain a product within a narrow temperature range, a method of making the same and a system. In particular, the present invention relates to temperature control elements and temperature control packages which can be readily manufactured from natural materials and be capable of being readily recycled. Phase Change Material (PCM) technology allows one to configure, vis-a-vis logistics, a mixture of PCM components, to maintain an insulated box or container over a prescribed period—typically from 12-120 hours—within a prescribed temperature range taking into account, the volume and weight of the particular good being transported, the nature of the container, the season, the climate likely to be encountered, the anticipated route (air/sea/land) container configuration and hemisphere. The present invention provides a cold chain system phase change material package, the package comprising a container and a phase change material; wherein the container includes a fibre-based absorbent material; and, wherein the phase change material is principally water.

### FIELD OF INVENTION

The present invention relates to temperature control packages, such as coolant packages that are used to maintain a product retained in a transport/storage package or container within a narrow temperature range, a method of making the same and a system. In particular, the present invention relates to temperature control elements and temperature control packages which can be readily manufactured from natural and/or ecologically sound materials and be capable of being easily and readily recycled.

### BACKGROUND TO THE INVENTION

In various fields of application, the transport of temperature sensitive products must be strictly observed. This holds in particular for a number of products (e.g. biotechnologically produced products containing proteins) produced by the pharmaceutical industry which are to be distributed to hospitals, pharmacies, etc. For such products, it is often a requirement that a continuous (uninterrupted) cold chain must be maintained from production until delivery of a product. A typical prescribed temperature range is 2° C. to 8° C. Equally, comestible agricultural produce, typically requires a constant temperature regime in transport and distribution systems. Transport boxes and containers that are employed are highly insulated; frequently the temperature in the transport boxes or containers used for the transport of the products can be monitored during transport, so that upon receipt of the container, the recipient may easily check whether or not the cold chain has been interrupted.

Active air-conditioned systems can be provided, but these can be expensive and require a power supply for ensuring operation of the system, by way of a battery pack, mains electrical supply, a diesel generator or similar. It will be appreciated that aircraft cargo systems need to be self-sufficient and connection with external power supplies can be inconvenient and oftentimes is simply not possible; diesel powered refrigeration units whilst fine on truck-based 40′ containers cannot typically be used within aircraft holds and the like. Accordingly, industry standard systems for logistics favour passive temperature control systems.

Typical passive temperature-control elements include specialized gel packs and specialized phase change materials. A phase change material (PCM) is a substance with a high heat of fusion which, melting and solidifying at a certain temperature, is capable of repeatedly storing and releasing large amounts of energy. Indeed, temperature-control elements such PCMs are typically employed for international travel and national travel alike. The high thermal capacities together with latent heat of absorption/dissipation associated with a phase change of said specialized temperature control elements are used to cool/heat the interior of the container when shipping temperature sensitive items, whereby to maintain a product within a particular temperature range. PCMs can also be applied for the cases in which cooling of perishable goods has to be extended. This may occur in cases of technical problems of cooling units and long holding periods before delivery. Of course, not all products need to be associated with the maintenance of a cold temperature and some phase change materials have, for example, high melting points and can be used for keeping warm pre-cooked food. Restaurants or delivery services take advantage of these materials.

PCM technology allows one to configure, vis-à-vis logistics, a mixture of PCM components, to maintain an insulated box or container over a prescribed period—typically from 12-120 hours—within a prescribed temperature range taking into account, the volume and weight of the particular good being transported, the nature of the container, the season, the climate likely to be encountered, the anticipated route (air/sea/land) container configuration and hemisphere. FIG. 1a shows an example of a cold chain transport carton 10, which is provided with six phase change material containers 12 arranged about a thermally sensitive load 14 as is disclosed in GB2566792 (Softbox Systems Ltd.), with FIG. 1b showing a single phase change material container 12.

A number of materials including aqueous salts, hydrates, paraffins, plant-based and animal-based fats and oils are used in the formulation of PCMs. An aqueous solution is one which water is the dominant compound in a system, and exists as a liquid. The amount of water molecules can change with a solution. In hydrates, that amount is often constant. However, heating both of these can often liberate water molecules, hydrates requiring significantly more energy. In aqueous solutions, water molecules are normally randomly arranged, but can be well-ordered around solute molecules or atoms, and make up the majority of molecules in the solution. Water-based PCMs, whilst comprising substantially of water include products such as a glycol or ethanol whereby to reduce the freezing point, permitting storage temperatures up to −30° C. Salt hydrate PCMs contain an inorganic salt and water and the melting point temperature range typically being between 8° C. and 90° C. The benefits of salt hydrates are favourable material costs, high latent melting heat, good thermal conductivity and non-combustible. FIGS. 1c & 1d show first and second perspective views of a type of cardboard box 13 that is used to contain plastic bags (not shown) filled with phase change fluids that can conveniently be made from a blank 15 as shown in FIG. 1e. Such phase change material containers 13 can be placed on the top of a load 14 of a container 16 shown in exploded view, per FIG. 1f or in a wall-hung temperature control system as shown in FIG. 1g.

In both solutions and hydrates, hydrogen bonding is the major force holding water molecules close to each other, or other components. It arises from hydrogen atoms being attracted towards oxygen or highly electronegative atoms in either the hydrate or solution. In rare cases of particular hydrates, the water molecules are covalently bound to a backbone, but when the compound is heated, fragments of the compound combine into water molecules, liberating water from it. A disadvantage may be that poor crystal formation makes salty hydrates more sensitive to supercooling, namely, the solidification of the material occurs at a temperature is lower than the actual freezing point. Disposal of the resultant miscible product can also result in difficulties in that the fluid cannot be disposed through normal waste water channels. A significant change in density—and therefore volume—occurs during supercooling and can cause problems. Even though a container might be sized to leave some additional space for the expansion/contraction of the PCM, the rapidity associated with a change of volume that can occur during supercooling might cause breakage or buckling of the container, giving rise to unnecessary disposal and/or damage arising from leaks, which might not be detected at the time of failure. Additionally, the shape of the container can distort, not only possibly making use within a receptacle impossible, the thermal characteristics will not the same as intended.

Paraffins, namely paraffin and wax derivatives of petroleum have a melting point temperature range comparable to that of salt hydrates. The latent melting heat of paraffins is reasonable and they do not have significant problems with supercooling. However, prices are linked to oil prices and are therefore not stable whilst a significant further disadvantage is that of disposal, since the fluid cannot be disposed through normal waste water channels. Oil-based phase change materials also have a tendency to diffuse or leach through plastics containers such as polyethylene unless the plastics container has been treated by a fluorination process. Additionally, like all fossil fuels, the extraction of petroleum has a big impact on the environment. A further, significant disadvantage is that of the flammability of the material, which makes its use illegal in certain buildings and difficult in certain air freighting solutions without special precautions being taken.

Plant-based PCMs are derived from plant oils and animal fat and provide a wide range of melting temperatures, lying in the range −30° C. to 150° C., with specific latent heat values being greater than salt hydrates and paraffins, thereby enabling greater space/weight efficiencies than salt hydrates and paraffins. The main disadvantage is that of the high price per kg that makes large-scale applications too expensive, whilst also suffering from the disposal issues present with paraffins.

A further disadvantage that is typically present in known phase change media is one of ballooning. Ballooning arises since the volume of water is a minimum at 4° C. (i.e. water has a density maximum that occurs at 4° C.) and that when a body is placed in a reduced temperature environment, the body will lose heat energy from the outside of the body, leaving the inner body at a higher temperature until temperature equalization has fully occurred. When undergoing a temperature reduction below the freezing point, it will be clear that as the volume of water continues to freeze, the inside will remain a liquid for longer, occupying a greater volume until the temperature of −4° C. is achieved—when it will expand. This results in what is ideally a flat-sided container “pillowing” or “ballooning” due to the fact that the volume of the central body of water/ice is greater than that provided by—or otherwise “permitted” by the encasement, container or surrounding ice. It is believed that ballooning can, at least in part, be exacerbated by super cooling.

Heat storage accumulators that are filled with PCMs are conveniently made from a plastics material—typically a high density polyethylene HDPE. However, such containers can suffer similar issues with regard to leaching of contents, through osmotic effects, as occurs with petroleum fuel tanks with automobiles unless surface treated by, for example, a fluorination process or similar, whereby the plastics walls are impermeable to phase change materials. It will be appreciated that these procedures will be followed in the manufacture of PCM containers, adding to the cost, where in an automated plant for the filling-in and sealing of heat storage accumulators, the moulding will be followed by a surface treatment prior to filling and capping by a production equipment. Today, a large proportion of recyclable materials are incinerated or otherwise improperly disposed of, ending up in landfills or even worse, the oceans of our planet. In a fully circular economy, the polymers employed for PCM containers should be recycled properly for reuse.

Problems associated with present-day PCM products can then be summarized as including disposal issues and supercooling include: the risk of not crystallizing and thus not fully releasing the stored heat particularly if the temperature of the heat transfer fluid (HTF) is close to a particular crystallization temperature; fluorination of plastics containers for certain types of PCM; variable density issues (ballooning); and, random nature of the phenomenon makes latent heat thermal energy storage (LHTES) difficult to control.

### OBJECT OF THE INVENTION

It is an object of the present invention to seek to remedy the deficiencies described above. The present invention seeks to provide an ecologically sound phase change material system, conveniently employing fewer artificial products. The present invention further seeks to provide a more ecologically sound phase change material container, with all or most products being conveniently disposed of using non-specialized compost and disposal routes. The present invention seeks to provide a method for producing an ecologically sound phase change materials and containers therefor. The present invention also seeks to provide a fully recyclable temperature control material, system of operation and production. The present invention also seeks to provide a temperature control material that maintains its shape, without excessive ballooning.

### STATEMENT OF INVENTION

In accordance with a general aspect of the invention, there is provided a latent heat storage medium comprising water with a cellulose fibre absorbent body retained within a flexible bag. Accordingly, in a basic form the present invention provides a latent heat storage medium that can be re-cycled and substantially removes issues arising from supercooling and any associated issues such as ballooning. The cellulose fibre absorbent body can comprise wood and/or plant cellulose fibre, conveniently derived from an air-laid manufacturing process or can comprise a sponge derived from a cellulose fibre sponge manufacturing process. The phase change material is water. It is believed that by having a non-supersaturated fibre body, supercooling effects are prevented. This provides a far greater degree of uniformity and it is believed that it can assist in reducing any ballooning of the container.

Conveniently, the PCM absorbent body is retained within a fibre based sheet material container, the container being formed from and sealed into a tube or tube-like shape, the tube or tube-like shape having an axis, is sealed across the axis to define a closed container.

The fibre based sheet material of the cold chain system pcm package can comprise wood/cellulose fibre. Preferably a fibre based sheet material of the container to the pcm package is provided with a plastics waterproof layer or coating. Conveniently, the plastics waterproof layer is applied as a hot-melt product, in amounts that do not preclude subsequent recycling through normal recycling centres. The container bag can also be manufactured from a plastics material that can easily be recycled.

The cold chain system pcm package can be prepared from a fibre based sheet material treated with a pliable mineral composite including a thermo-formable bonding agent sufficient to enable a storage article containing the mineral layer to be formed into a selected shape via a thermo-forming process and/or pressure/vacuum forming process. To assist in handling of raw materials, the air-laid fibre or sponge insert can be compressed prior to use.

To reduce handling problems, it is convenient to ensure that the insert is not saturated or supersaturated with the PCM fluid; conveniently, the insert is, for example, 30-95% saturated with water, preferably 50%. Providing a good safety margin, reducing damage through leaks upon damage, the present invention can provide an uncomplicated, wieldy PCM solution as a product, system and method of use. A biocide could be provided within the fluid to prevent bio-growth within the phase change material container. In particular, when employing air-laid fibre which has been mechanically formed into a fluted form, which has subsequently been folded into a multiple-layered fluid retention volume, it has been found that that that when water is introduced into a central zone, fluid tends to be preferentially retained along the flutes, spreading less slowly with respect to a direction orthogonal to the flute direction.

A biocide could be provided within the fluid to prevent bio-growth within the phase change material container. In particular, when employing air-laid fibre which has been mechanically formed into a fluted form, which has subsequently been folded into a multiple-layered fluid retention volume, it has been found that that that when water is introduced into a central zone, fluid tends to be preferentially retained along the flutes, spreading less slowly with respect to a direction orthogonal to the flute direction.

To address the needs of an increasing environmental consciousness, the issues of disposable packaging have become increasingly stringent. Replacing environmental-unfriendly materials such as plastic, styrofoam, etc. by biodegradable materials is a present trend to reduce the burden on the Earth's environment. However, since paper materials are not in themselves waterproof and greaseproof, food contact surfaces of paper-based containers are usually coated by an insulation/barrier layer, such as wax, or a PE film, to provide waterproof and greaseproof properties, but these can inhibit recycling. The present invention teaches of a solution to mitigate the recycling and re-use of plastics and biodegradable issues in the field of cold-chain logistics.

### BRIEF DESCRIPTION OF THE FIGURES

For a better understanding of the present invention, reference will now be made, by way of example only, to the Figures as shown in the accompanying drawing sheets, wherein:—

FIGS. 1a-1b illustrate a prior art cold chain carton and phase change material container;

FIGS. 1c-1g illustrate a prior art cold chain phase change material carton and how it can be placed within a palletized container;

FIG. 2a-2e illustrate a phase change packet in accordance with a first aspect of the invention;

FIG. 2f-2g illustrate a phase change packet in accordance with a first aspect of the invention;

FIG. 3a illustrates two inner phase change packet lying one on top of another;

FIG. 3b shows a plan view of an inner phase change packet;

FIGS. 4i & 4ii show a cellulose foam in a states of compression and in an expanded state operably employed for retention of water as a PCM fluid;

FIG. 5 shows an outline process for manufacturing a coated waterproof paper;

FIG. 6 shows an arrangement of two collocated phase change envelopes under test;

FIGS. 6a-6d are graphical representations of four freeze-thaw tests performed with respect to a water coolant;

FIGS. 7a-7c are graphical representations of four freeze-thaw tests;

FIGS. 8a-8c are detailed representations of solidification phase results; and,

FIGS. 9a-9c are detailed representations of liquefaction phase results.

### DETAILED DESCRIPTION OF THE PREFERRED EMBODIMENTS

There will now be described, by way of example only, the best mode contemplated by the inventor for carrying out the present invention. In the following description, numerous specific details are set out in order to provide a complete understanding to the present invention. It will be apparent to those skilled in the art, that the present invention may be put into practice with variations of the specific.

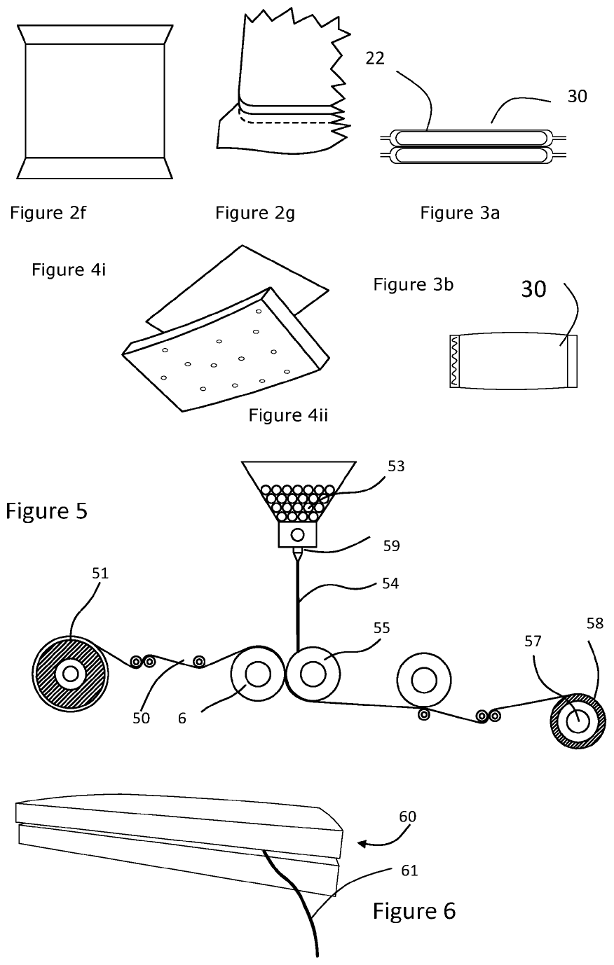

Referring now to FIG. 2a, there is shown a first example of a phase change material packet 20 in accordance with a first aspect of the invention. The primary phase change material is water (not indicated), provided as a purified material, such that it has known thermal properties, in particular its specific heat capacity and its latent heat of freezing. It will be appreciated that additives can be provided to affect such qualities, but ease of disposal tends to be comprised by the use of several known water-based PCM solutions. Notwithstanding this, Applicants have discovered that wood pulp or cellulosic fibre can be provided, conveniently in laminar sheet form 22, to enable a substantially saturated wood pulp body to be formed. The wood pulp, as expected does not affect the temperature at which phase change occurs, but more importantly, reduces the possibility of differences arising from non-linear cooling effects such as supercooling. Moreover, it is believed that the presence of the fibres enables phase transitions from liquid to solid to be accurately characterized and determined, with surprising results enabling more accurate thermographic modelling of phase change media in accordance with the present invention. It is believed that the fibres provide an effect not too dissimilar to the Mpemba effect.

It has been found that using typical wood pulp, 10 ml of water can be retained and associated with each gramme of wood pulp. The dimensions of the packet 20 can be varied, but it has been found convenient to have the size in general correspondence with known PCM containers, with dimensions of the order of 17×12×4 cm. This enables simpler calculations when replacing or exchanging phase change media products. It is believed that latent heat of the wood pulp and the water together is greater than the latent heat of water alone, whereby to provide a phase change material with altered—yet controlled—characteristics. FIG. 2b shows the completed bag of FIG. 2a in plan view with sealed edges 24 at either end of the bag. The bag can be formed from paper that has been made waterproof as shall be discussed in detail below. FIG. 2c shows a cross-section along lines A-A per FIG. 2a through a phase change material packet of the type shown above, indicating a number of wood pulp sheets 22 arranged in layers. FIG. 2d shows a plan view of an initial paper bag embodiment 25, wherein adhesives were applied to a waterproof paper; FIG. 2e shows a perspective view of embodiment 25 including a folded section 26. FIG. 2f shows a frozen pcm comprising a plastics bag enclosing an air-laid wood pulp “biscuit” which had been compressed prior to absorption by water. FIG. 2g shows an absence of ice formation which might otherwise occur if the fluff-pulp biscuit is super saturated. It has been noted that air-laid fibre, tends to be arranged in a random fashion and the structure is isotropic, but can be embossed to provide a fluted structure not too dissimilar in appearance to single-sided corrugated cardboard due to the method of forming the wood pulp. In the case of Air-laid fibre, the raw material is conveniently long-fibered softwood fluff pulp in roll form. The pulp is defibred in a hammer mill. Defibration is the process of freeing the fibres from each other before entering an air-laid machine. When this fluted product is subsequently arranged in layers, it has been noted that absorption of fluid is more favourable along the axis of the flutes as opposed to a direction across the flutes. This fluid absorption process has been taken advantage of in that, a multiple-layered fluid retention volume has been shown to permit passage of an amount PCM water faster along the flutes, whereby, in manufacturing and initial installation absorption is more controlled if the direction of flute is perpendicular to a major plane of a generally rectangular cuboid pcm package.

In view of the fragile nature of the wood pulp and to assist in handling, transport and storage, in accordance with a second aspect of the invention, it has been found convenient to arrange 2-5 sheets of wood pulp in layered groups to enclose the groups of layers of wood pulp within a small, porous, sealed bag or packet whereby to provide structural stability to the wood pulp sheet material, as is shown in FIGS. 3a, 3b to resemble a bag in the style of a tea bag, as is known. The porous nature of the bag permits full water absorption of the fluff pulp and can conveniently be made from a paper being a blend of abaca cellulose fibres, whereby to maintain an overall recyclability of the product yet provide a degree of strength and usability to the wood pulp. Notwithstanding this, from a manufacturing costs point of view, it is desirable not to introduce needless process to shape the material into a particular form, if possible. Applicant Company has been using pulp products from SCA of Sweden, which provides several types of pulp, including chemical thermomechanical pulp (CTMP) produced from a number of different species of trees, including spruce, birch, aspen and pine whereby to produce a pulp with high freeness and bulk yet with a low shive and fines content, which has been found to be more absorbent than typical wood pulp. In particular, Applicant Company has found that SCA's pulp sold under the 6035 brand has worked well, using 150 g of fibre per litre of water—so that it is 50% saturated (15% by weight). It has been found to perform well; the phase change material does not become easily over saturated when compressed, providing a useful degree of tolerance when being handled. Whilst this brand provides fibre lengths of are nominally 1.9 mm long, with diameters of 10-30 μm it will be appreciated that fibre lengths of 5-0.5 mm can be usefully employed, but limitations may arise in the handling of the stuff in production processes and the like. Whilst 150 g of fibre per litre of water is preferred, other values of 10-20% by weight have also been found to provide goods results.

The porous paper can be formed into a tube using known procedures wherein, in a manufacturing process, a paper provided from a reel having a width with first and second edges at either side is sealed together along the first and second width-wise edges whereby to define a tubular element with a longitudinal axis. The sealing procedure is conveniently performed by way of a continuous process using at least one of heat, pressure, corn starch and plastics sealant. Corn starch adhesive technology is known from the production of modern-day tea-bags, whilst biodegradable plastics sealants are presently being developed. In a typical production process, to form a bag product, once the axial seams have been sealed, a first perpendicular seal is made across the axis of the tubular element whereby to define a first end of a bag, to permit placement of cellulose fibres. Indeed, such bags can be manufactured in a chlorine-free, unbleached manufacturing process, enabling the cellulose fibres and bags to be fully recyclable and/or biodegradable.

As an alternative to the use of wood pulp/cellulose pulp, in accordance with a further aspect of the invention, there is provided a phase change material package wherein a cellulose sponge is employed to define an absorption body to retain the water. The cellulose sponge can be placed within bags that are initially or subsequently filled with water, noting that the procedure of allowing the sponge to absorb water is facilitated by the use of an initially dried sponge. FIG. 4i shows an exemplary cellulose sponge in a dry compressed state and FIG. 4ii shows a similar cellulose sponges in a semi-saturated state. Weight densities of the order of 10-20% have also been found to be suitable.

To complete the manufacturing process, the cellulose fibres, including any porous bags employed to contain the same, or compressed cellulose foam and water need to be placed within a waterproof bag. In a process not too dissimilar to packaging of the porous, bags as described above, waterproof paper needs to be formed into a general tube-like shape using known procedures wherein, in a manufacturing process, a length of waterproof paper is provided from a reel, the length of paper having a width with first and second edges at either side which are sealed together along the first and second width-wise edges whereby to define a tubular element with a longitudinal axis. A method of making recyclable waterproof paper is discussed below, but the plastics film-paper adhesion properties may not be sufficient to enable a secure seal by the use of an adhesive alone, since the waterproof plastics layer could be compromised and delamination occur. Nonetheless, a sealing procedure can conveniently performed by way of a continuous process using at least one of heat, pressure, adhesive and plastics sealant. As above, in a typical production process to form a bag product, once the axial seams have been sealed, a first perpendicular seal is made across the axis of the tubular element whereby to define a first end of a bag, to permit placement of cellulose fibres plus water in a substantially reduced pressure gaseous environment, followed by a second sealing operation whereby a closed phase change material container in the general form of a bag is produced.

The embodiments above have been presented as fibrous fluff/cellulose materials contained within a recyclable plastics bag. Nonetheless, it will be appreciated that other forms of biodegradable waterproof containers could be provided, such as those made from thermo-moulded and glued paper, which has been treated with, for example, plastics and mineral products. It will be appreciated that other forms of providing waterproof cellulose packages can be provided, for example it has also been found possible to use containers made from moulded pulp by means of a mechanized papier-mâché, which pulp has been made waterproof by using plastics coatings as disclosed in relation to the waterproof paper. Fibre-based recyclable waterproof containers and methods for the construction thereof are known from a number of suppliers, including Plastiroll Oy Ltd (Walkie).

In yet a further aspect in accordance with the invention, a mineral composite structure suitable for forming storage articles can be provided as a receptacle for pulp fibre or cellulose sponge materials, the structure comprising an extruded or blown mineral with a bonding agent comprising a mix of polymers, including polypropylene. The mineral-containing structure is sized and manufactured such that it is capable of being shaped to form a layer of a composite structure with one or more other layers of fibre and a waterproof recyclable layer such as a poly-lactic acid coating. Conveniently, the composite structure is formed into the shape of a box or carton for placement in a shipper or other form of container. The composite structure can also contain woven and non-woven reinforcing flat webbed materials as a layer, which layer has been found to add significant burst strength and tear resistance. Notwithstanding this, the results were not as good as plasticized sheets as described above.

Applicant Company has performed tests on the air-laid embossed product (in its dry state) and it compares favourably with polystyrene vis-à-vis its thermal insulation properties. As will be appreciated, the specific heat capacity of a water-saturated cellulose fibres packet is not too dissimilar to that of pure water. However, it has become apparent that when a cellulose fibre-fluff pulp packet in accordance with the present invention undergoes a phase change, notably from liquid to solid, the process occurs uniformly over a narrow temperature range, given that a temperature gradient will exist across a distance over a body of water, from the exterior, towards the centre thereof. It is believed that the fibres present in the phase change packet act to assist in a transfer of heat. The characterizations of cellulose fibres relevant to the present issue include: wicking speed, absorbency. Absorbent products made with such fibres exhibit a high degree of fluid retention, liquid distribution (wicking) and pad integrity. This has a result of extending the liquefaction phase change period with regard to known phase change materials.

Air-laid non-woven wood pulp is presently used in a variety of absorbent applications, including baby nappies and adult incontinence pads, feminine hygiene products, pet pads and food-grade absorbent pads and the technology is reasonably well developed. Wood pulp can be produced by defibration of cellulose for incorporation into absorbent products and are made from coarse, bulky, long fibres. Some types of pulp such as fluff pulp are prepared by chemically treating cellulosic materials, such as softwoods and hardwoods, to remove their lignin fraction and produce a cellulosic pulp suitable for making paper and related non-woven products. In certain pulping processes, a cellulosic source such as wood chips is digested with alkaline liquor containing sodium hydroxide and sodium sulphide to effect lignin removal, but such lignin removal is not necessary with regard to the present invention and enables a simpler purely mechanical wood pulp to be used. Wood pulp can be produced in the form of roll pulp, but also can be sold in sheet form as bales (hereinafter both referred to a dried fluff pulp sheets). Conventional wood pulp sheet is manufactured, following the chemical pulping operation, by forming the pulp into a sheet or non-woven mat by any one of several well-known wet-forming processes typified by the conventional Fourdrinier process. In a first step, bleached chemical pulp slurry is deposited upon a screen (or “wire”) to form a mat or web of pulp fibre. This step, known in its initial stage as formation, is usually accomplished by passing an aqueous dispersion of a low concentration of pulp (e.g., 0.5% to 1% by weight solids is typical) over the screen. This screen, assisted in certain situations by vacuum or suction, increases the consistency of the mat or web to approximately 20 to 35 weight % solids.

In a second step, the mat or web is compressed or squeezed in a “press section” to remove additional water. This is usually accomplished by felt presses, a series of rollers each having a felted band for contact with the mat or web. These presses remove additional free water and some capillary water, thus resulting in an increase in consistency of the mat or web to a range of about 30 to 60 weight %. As is well known, in making wood pulp sheet, less pressure is applied in this portion of the process than normally would be encountered in conventional paper-making, thus less water is removed in this section. Less pressing is done so as to facilitate subsequent comminution of the wood pulp sheet to the defibred, fluffed pulp.

In a third step, the pulp sheet is then dried in a dryer section. Because a reduced amount of water was removed in the press section, more moisture must be removed from the sheet in the dryer section than generally is necessary in paper-making. In the drier section, the remaining water content of the pulp sheet is reduced to obtain a pulp consistency which typically ranges between about 88 to 97 weight % (3 to 12 weight % moisture), more usually between 90 to 94 weight % (6 to 10 weight % moisture). For use in absorbent products, the sheets formed in this manner are thereafter comminuted using a variety of known techniques and machines such as hammer mills, as briefly indicated above.

The pulp slurry typically includes cellulose fibres such as chemically digested wood pulp fibres as its main component and may also include as a minor component, mechanical wood pulp and synthetic or other non-cellulose fibres as part of the slurry. In the broad aspects of the present invention, it is also contemplated that the pulp may be treated with bond-inhibiting chemical substances (so-called debonders), chemical softeners, or other chemical additives during preparation of the cellulose fibre pulp sheet to alter processing or aesthetic characteristics of the finished fluff pulp and the absorbent products made from said cellulose fibre pulp. A bleaching process can also be performed to ensure additional lignin removal, and increase the whiteness and brightness of the pulp to enhance commercial acceptance. The addition of such chemicals is normally effected by adding the chemical to the pulp prior to sheet formation or by spraying the pulp after the formation of the non-woven sheet or mat and sometimes during initial mechanical dewatering. Applicants have employed air-laid pulp that has been compressed, provided by M&J Airlaid Products of Denmark.

As an alternative to the use of fibres, cellulose sponge has also been found to be suitable. In a similar fashion to pulp fibre, cellulose fibre can be obtained from wood and plant matter. For many reasons it can be a big advantage to compress viscose sponge material. Generally the compressing delivers many more advantages, like cost savings in storage and logistics. Viscose sponge foam typically employ presses with up to 250 tonnes of power to compress the sponge. Compressed sponges are also known as expanding, swelling or pop-up sponges and are formed using dry cellulose sponges which are compressed under high pressure and heat. On contact with liquids they expand to their original size and have the same properties as un-pressed sponge from thereon. This means that they offer specific advantages for further processing and applications, including the simplification of storage and distribution of the material for subsequent use in manufacture, noting that the liquid absorption factor of a compressed sponge is up to 20 times its own weight with compressed sheet easily formed in sizes of up to around 850×800 mm. The sponge can also be identified by the use of printing and dyes to indicate specific capabilities. These factors, together with the ability of the sponge to be produced in square, round, oval or of individual forms mean that the sponge can be specially adapted to ultimate application and also enable the production of custom phase change material containers at the premises of an ultimate distribution company, simply enabling custom packets to be made and deployed.

Nonetheless, some biodegradable materials such as starch, and polylactic acid, etc. has been developed as lamination films to provide a waterproof layer for a fibre material such as paper. With reference to FIG. 5, there is shown an operational scheme of a known paper coating process, where a paper 50, initially wound around reel 51 is conveniently coated by, for example, polylactic acid. Polylactic acid coated paper is simply manufactured by an extrusion process. In this method, solid polylactic acid feed stock 53 is heated to form a hot molten yet viscous polylactic acid resin 54 which is directly extruded onto a cooling roller 55, and then applied to paper 50, the paper 50 passing through a small space that exists between the cooling roller 55 and an opposing spaced-apart pressure roller 56. Consequently, the paper 50 is coated by a layer of polylactic acid resin to form a waterproof film thereon and the paper with waterproof film 58 is collected by a roller 57. The biodegradable waterproof paper is provided with a polylactic acid polylactic acid resin film coated on at least one surface of a paper, the polylactic acid partially infiltrating within the fibres of the paper. The polylactic acid resin is preferably formed from a stock solution includes 1-85 wt % of polylactic acid, and 15-99 wt % of a solvent.

The level of an output of the polylactic acid resin 54 is controlled by an extruding element 59, the amount of the polylactic acid resin 54 output from the extruding machine 30 is applied in a uniform fashion. Furthermore, since the PLA coated film is laminated onto the paper surface by heat and pressure, the only adhesion strength between the polylactic acid film and the paper tends to be weak, which is of benefit in a subsequent recycle produce. Once the waterproof and biodegradable paper has been made, it will be clear that it can be folded and sealed using suitable glues, as are known. It will also be appreciated that polylactic acid is but one suitable biodegradable resin and that the method of applying the polylactic acid is but one method for providing such a film. Applicant Company, having conducted extensive tests, has determined acceptable moisture vapour transmission rates of less than 5% over six months can be provided by the use of a 30 gsm polyethylene coating upon a 70 gsm paper—as can be supplied by a number of paper companies, such as Mondi—from whom their Mondi 70/30 coated paper sheet satisfied such a requirement, noting that this paper was developed to be capable of recycling within present recycling contamination limits. Moisture vapour transmission rates (MVTR) were conducted by Applicant Company and were carried out in a chamber at 23 C and 40% Relative humidity, we have set a bench at a loss rate of 5% of the pack weight, typically being sized in 1 Kg units. It will be noted that no fluorination of the polyethylene was found to be necessary, as would be the case if known oil based PCMS, such as paraffin were to be employed.

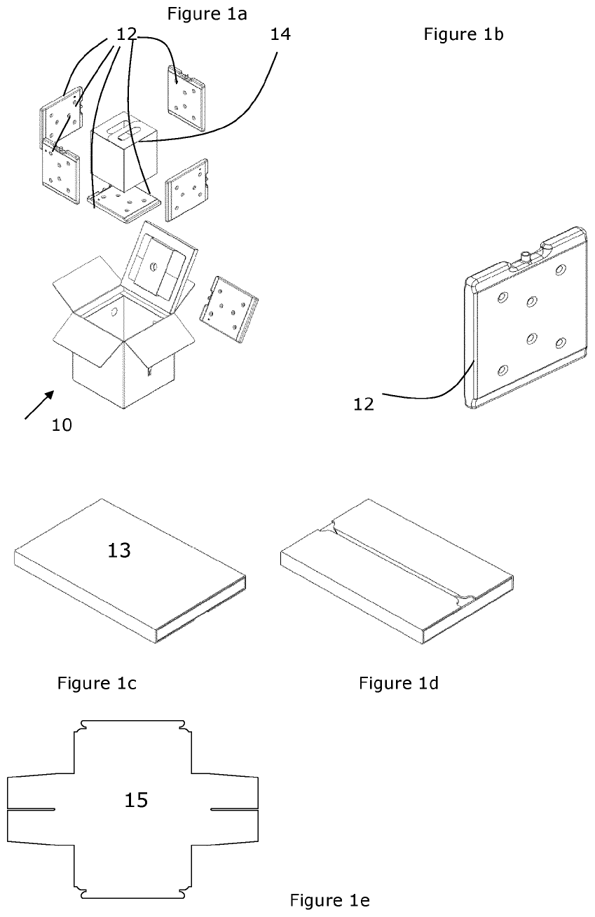

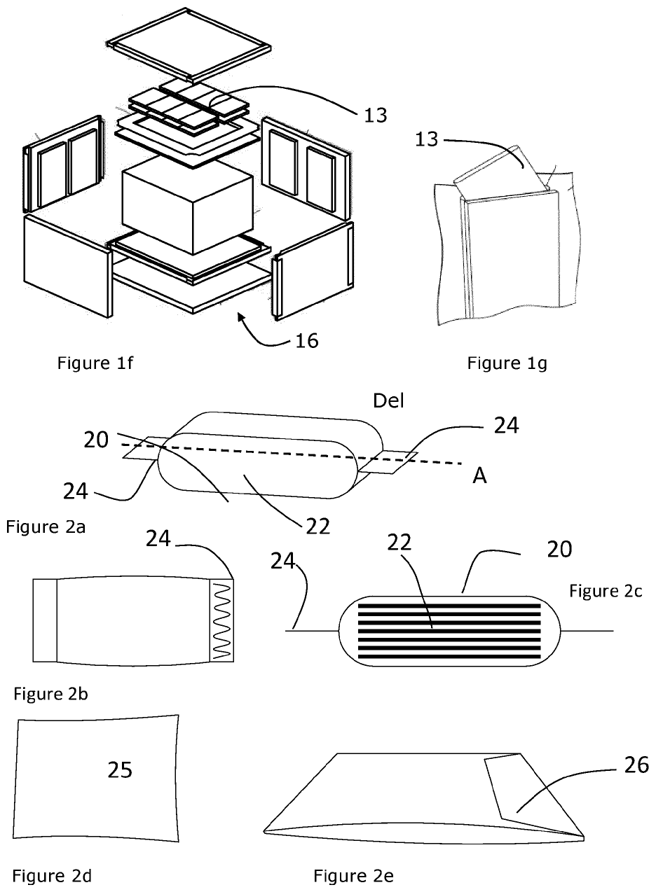

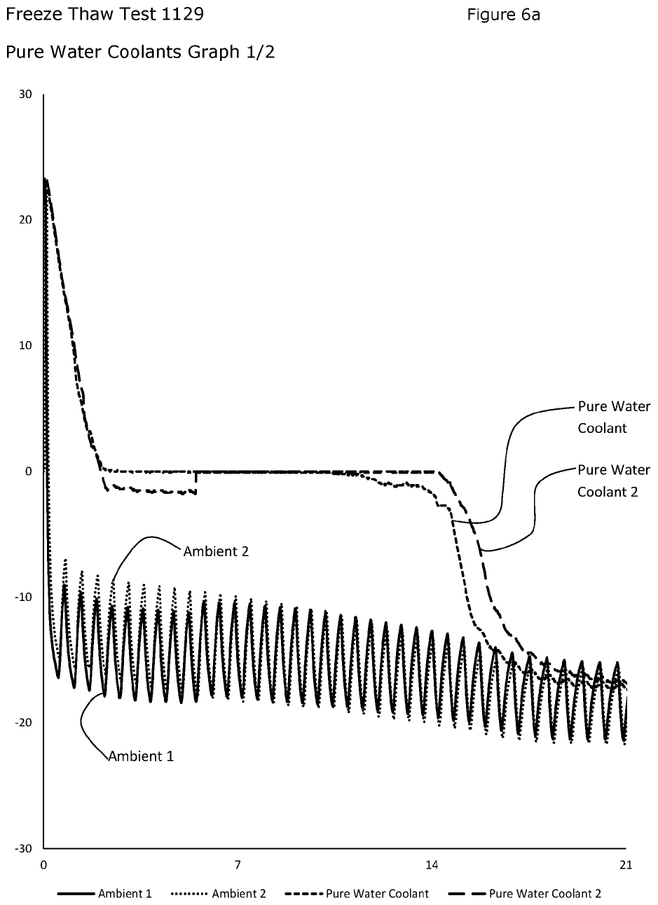

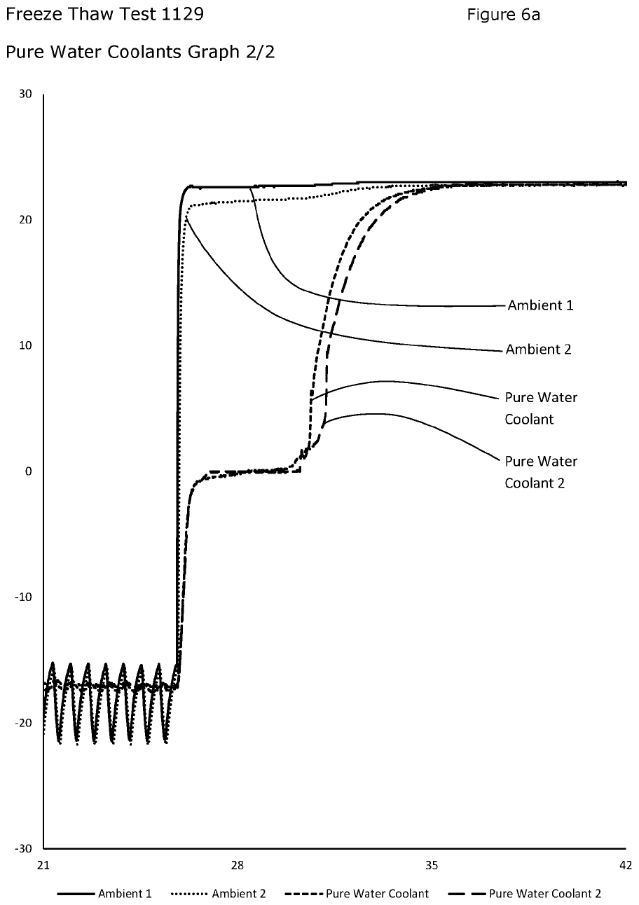

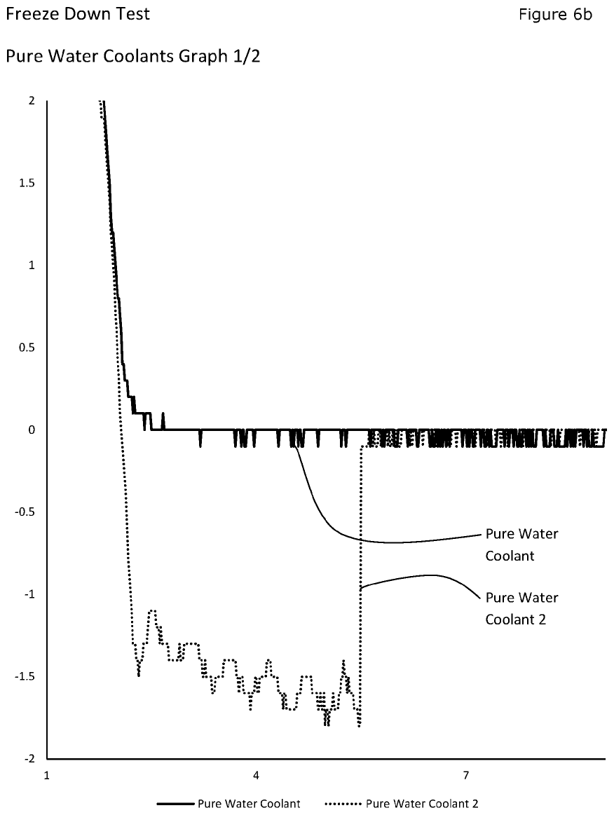

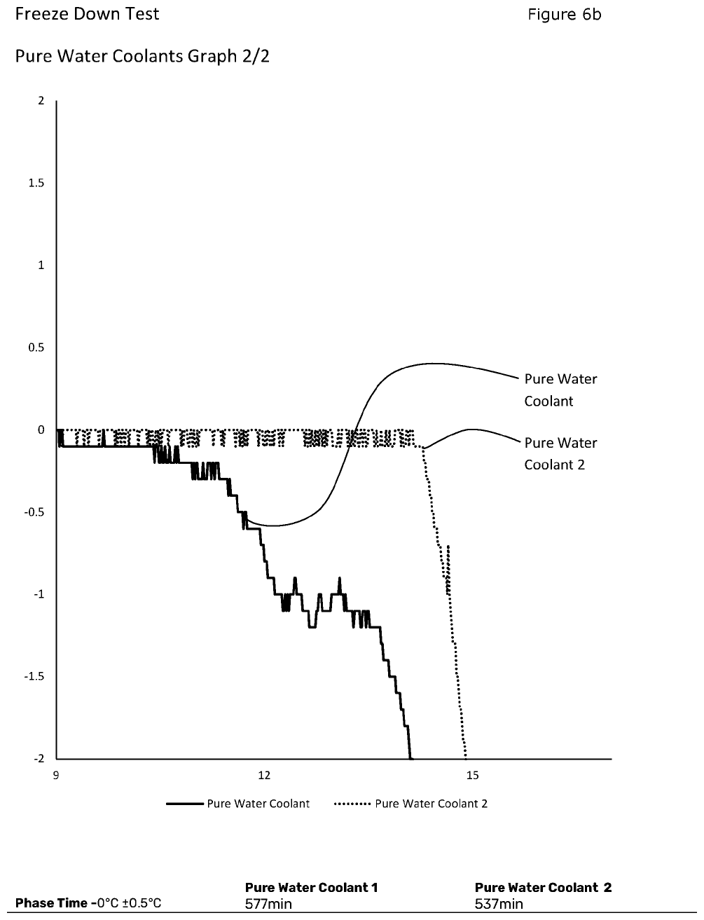

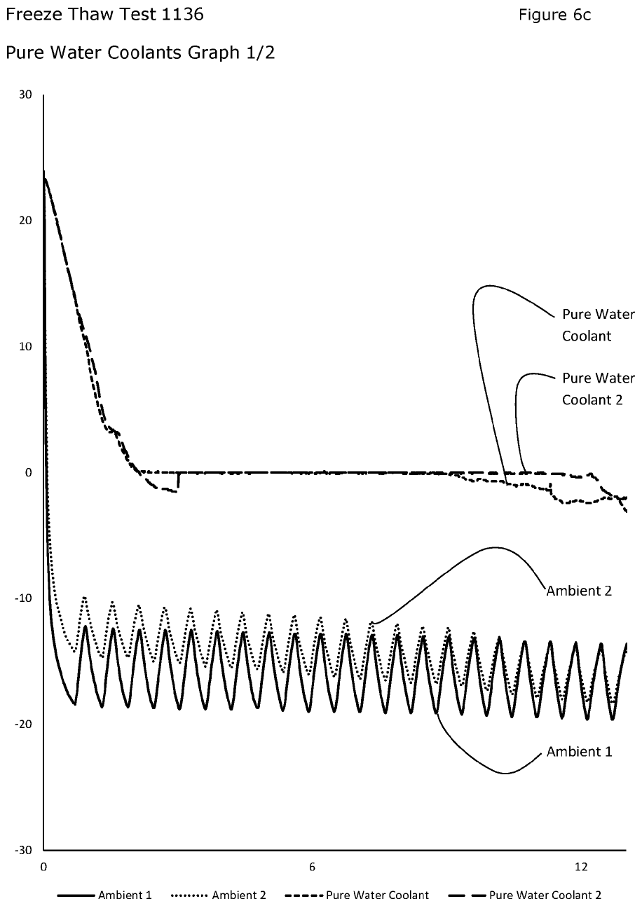

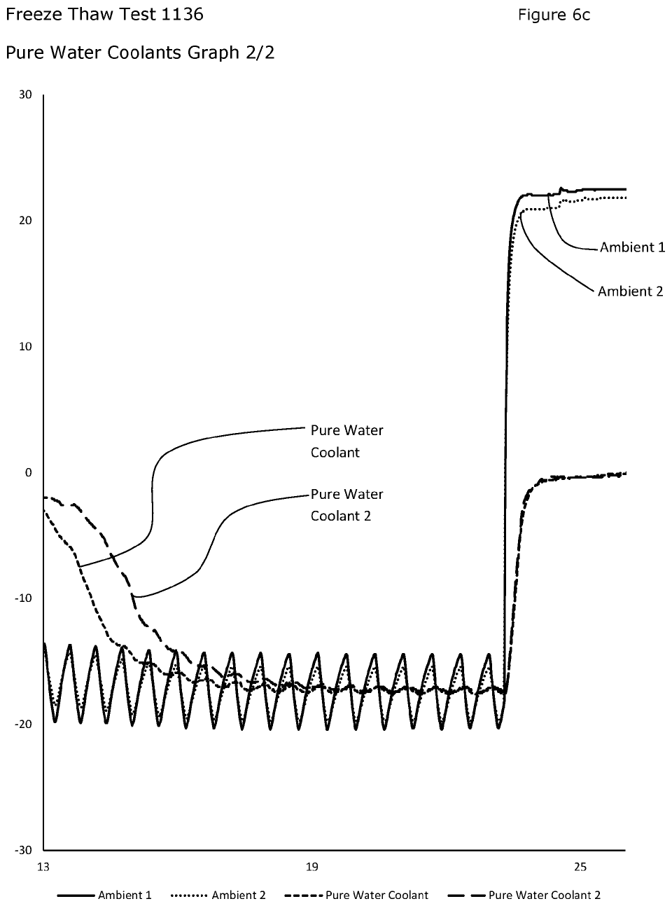

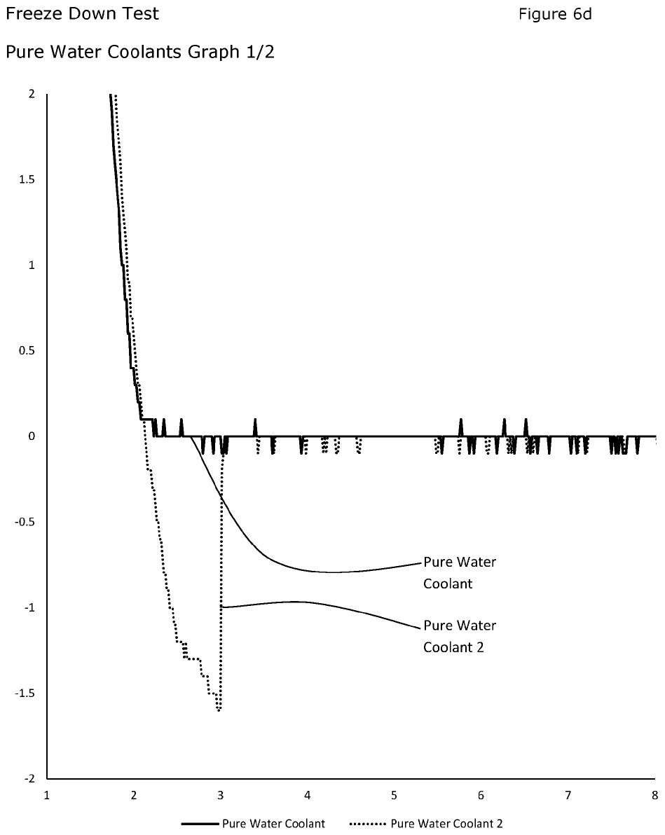

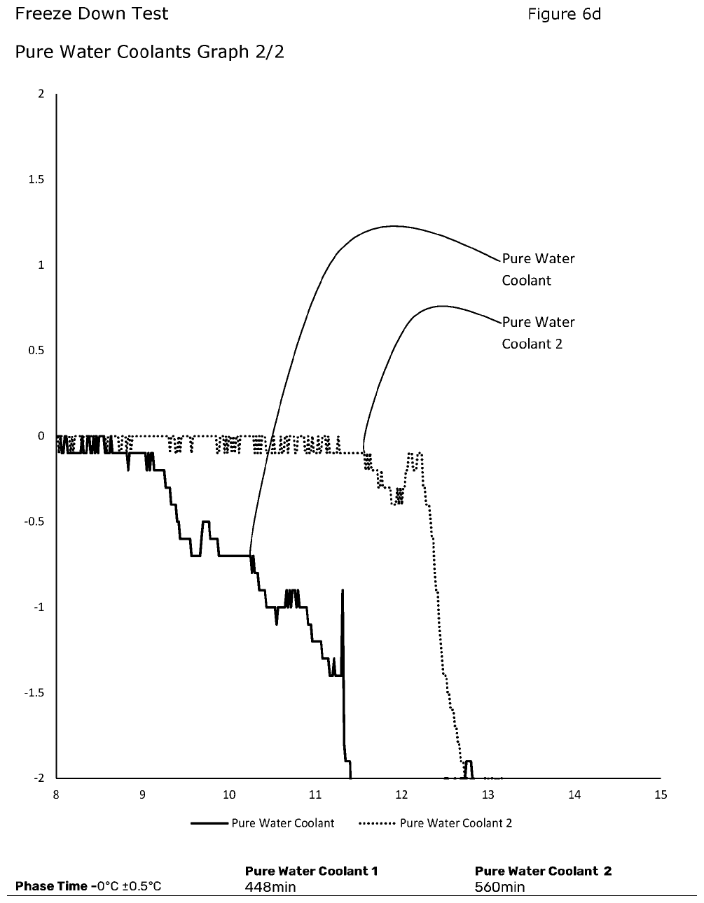

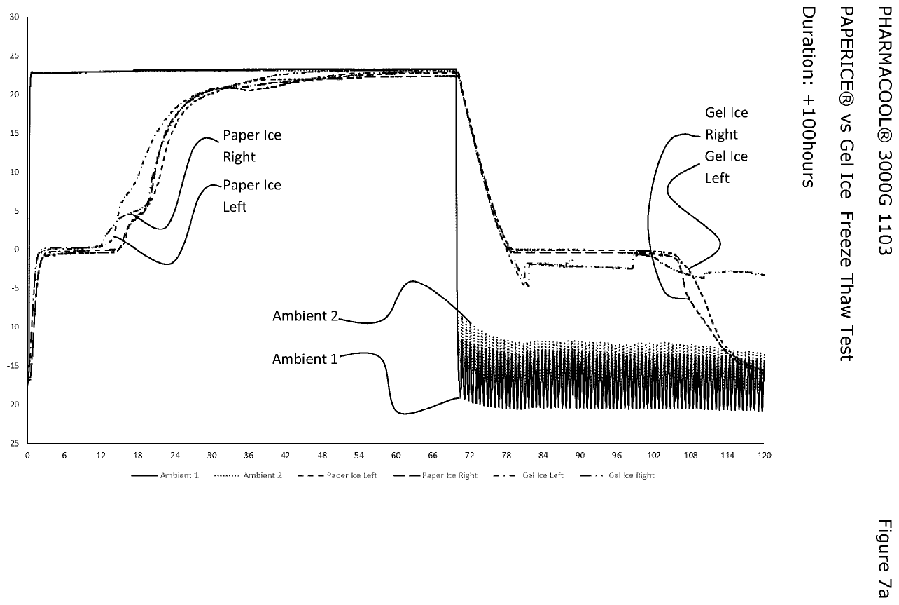

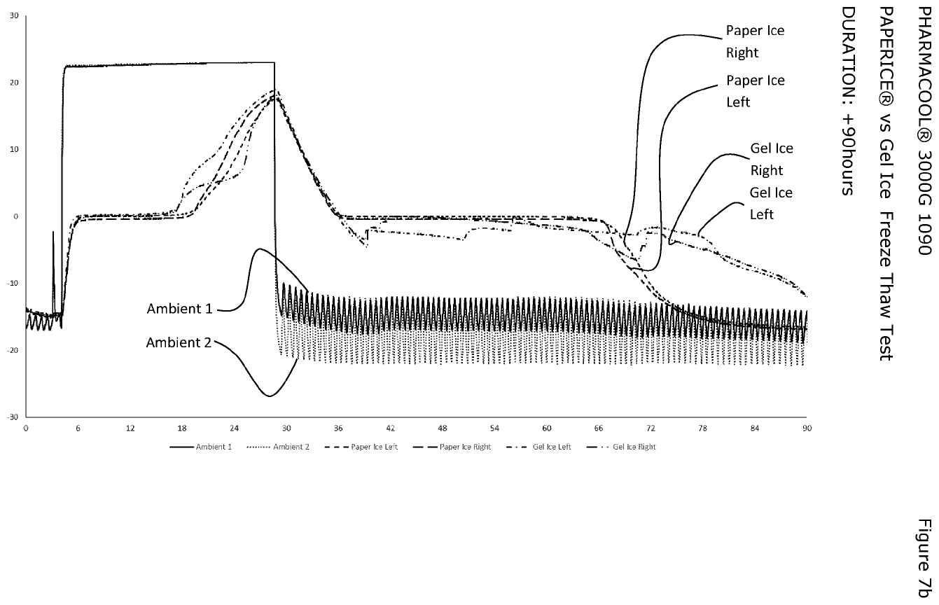

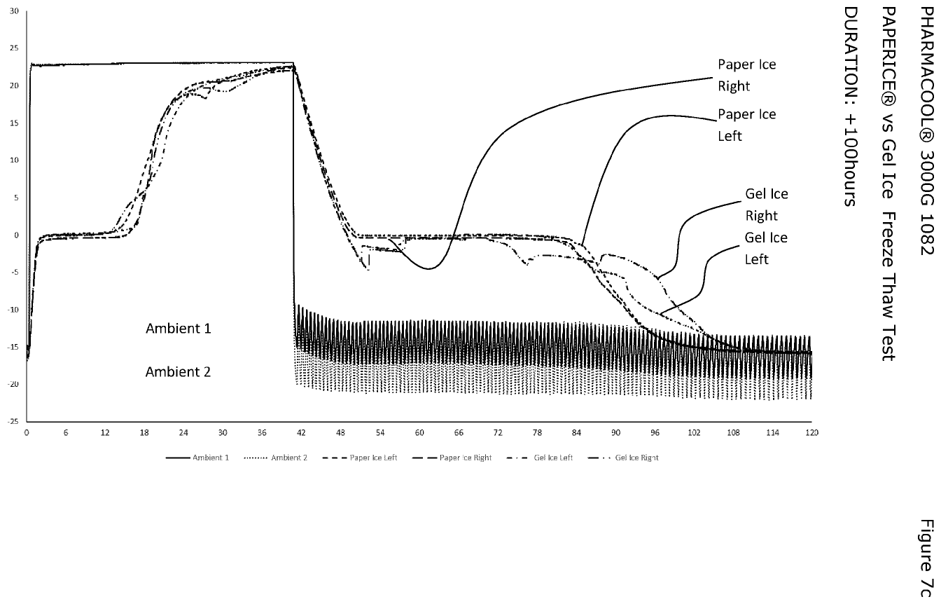

Referring now to FIG. 6, there is shown an arrangement of two phase change envelopes 60 as discussed with reference to FIGS. 1c-1e is used as a basic test system under test, with a main face of each of the two phase change envelopes facing each other in an abutting relationship, with a number of thermometer leads 61 being coupled to measurement equipment. The phase change envelopes are marketed under the PHARMA-COOL® trade mark by Applicant Company and are placed in an environmental test chamber during testing, with regard to an adjustment period, to normalize test procedure environmental conditions. The PHARMACOOL® packets have a cardboard container surrounding three packages containing phase change materials of temperature control, each of the packages comprising a 1000 g in weight. In the freeze/thaw test, the phase change envelopes comprised an example of phase change material in accordance with the invention, whilst a comparison was made with respect to a known gel mixture, namely a water solution with 4% by volume polyacrylate. The two envelopes were joined with double sided tape with two probes arranged therebetween, the different examples having the test probes being placed in, respectively, the same positions.

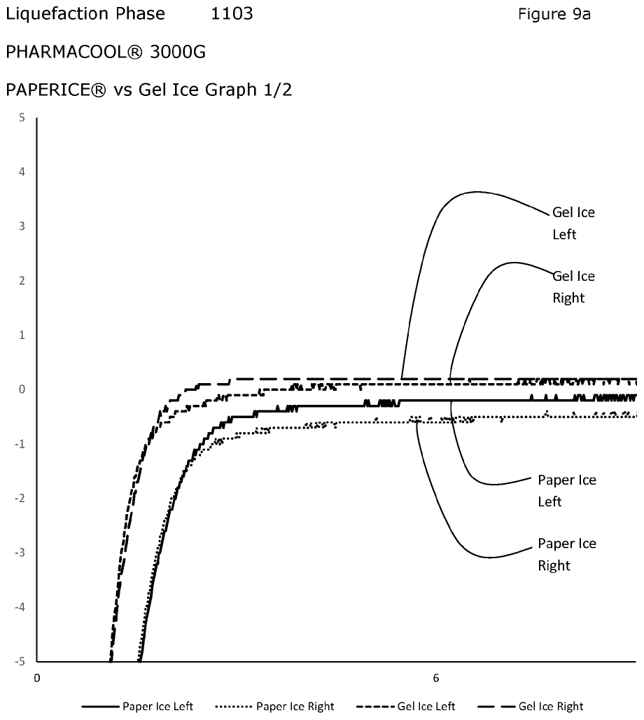

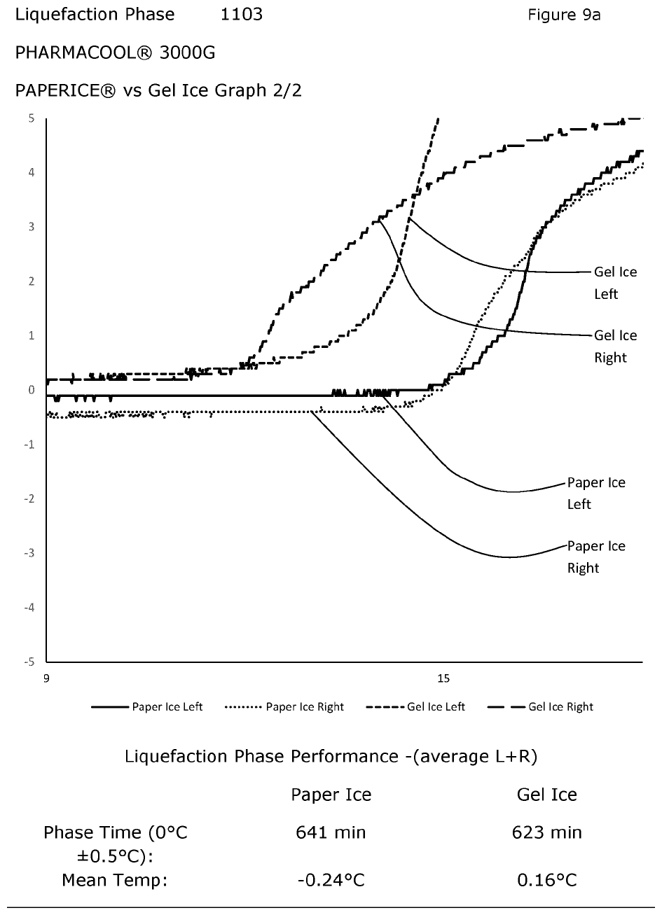

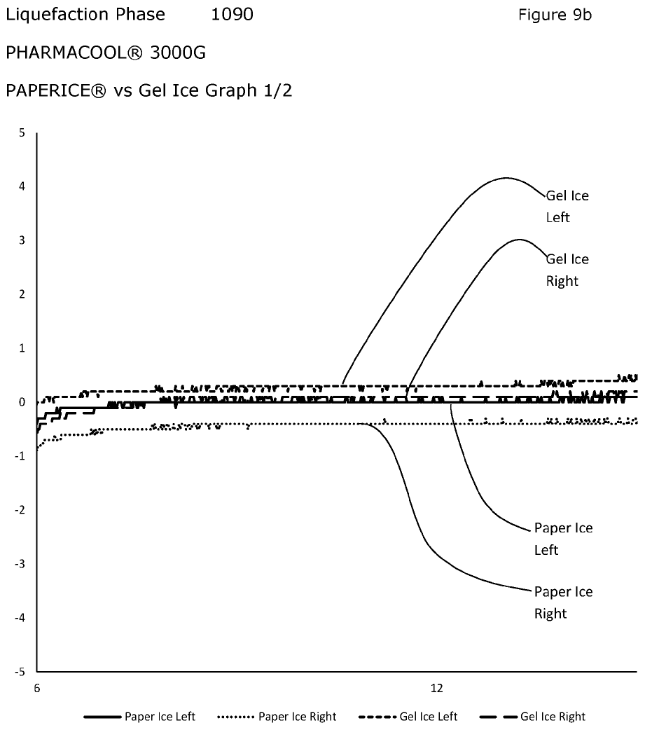

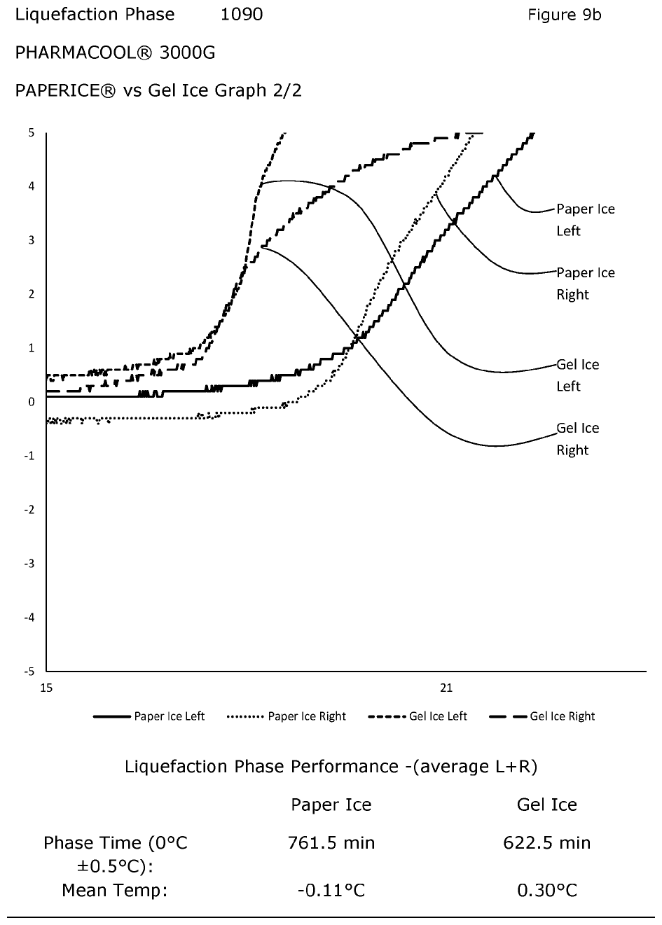

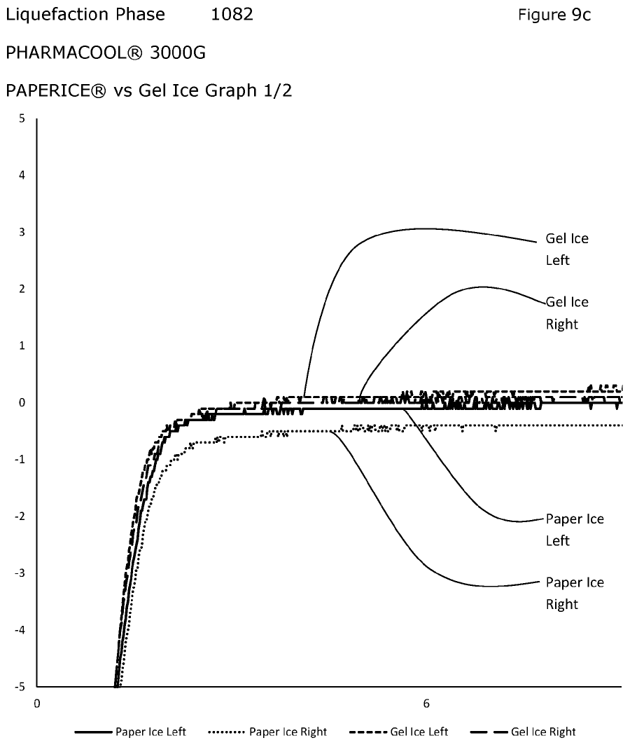

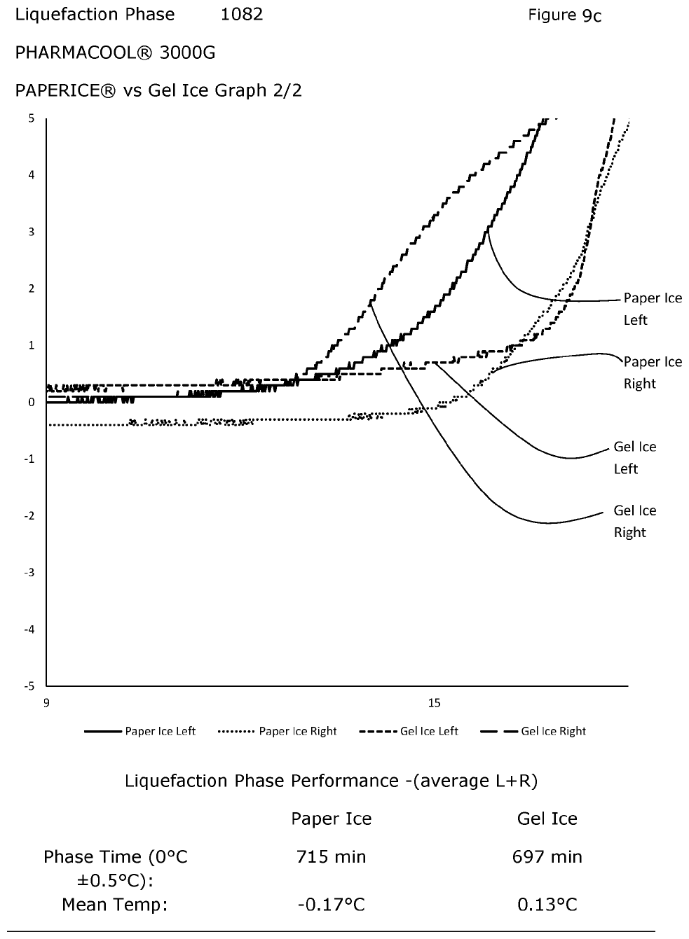

FIGS. 6a-6d show results in respect of a control system, where 500 g water only coolant bags were arranged in a freeze-thaw test for a periods of 42 and 24 hours, with the temperature starting at 23° C., being reduced to −18° C. and then being raised to 23° C. It can be seen that in each of the tests, a period of supercooling was observed, for periods of approximately four hours and one hour, during hours 2-5.5 as shown and hours 1-3 in FIGS. 9b and 9d, respectively, during average 0.0±0.5° C. phase transition times of 547 and 504 minutes.

For each test performed with respect to the present invention, a detailed analysis was performed in respect of the solidification phase and of the liquefaction phases. The temperature of the environment test chamber was varied from −18° C. to +23° C., and the duration for the freeze-thaw tests was undertaken over a period of 100 hours, the temperature of the chamber and the phase change envelopes being initially −15° C. with the chamber temperature being increased to 23° C.; after 40 hours the test chamber temperature was brought down to the initial temperature of −15° C.

| | |
|---|---|
| Test 7a: Test Duration: +100 hours; | Type: Thaw-Freeze |
| Test 7b: Test Duration: +90 hours; | Type: Thaw-Freeze |
| Test 7c: Test Duration: +100 hours; | Type: Thaw-Freeze |

Test results 8a-8c relate to the specific graphical representations of solidification phase results and FIGS. 9a-9c are detailed graphical representations of liquefaction phase results. The results clearly indicate that the present invention provides no effective supercooling—i.e. the temperature has decreased without a phase change and a longer period of constant temperature during a period of temperature reduction.

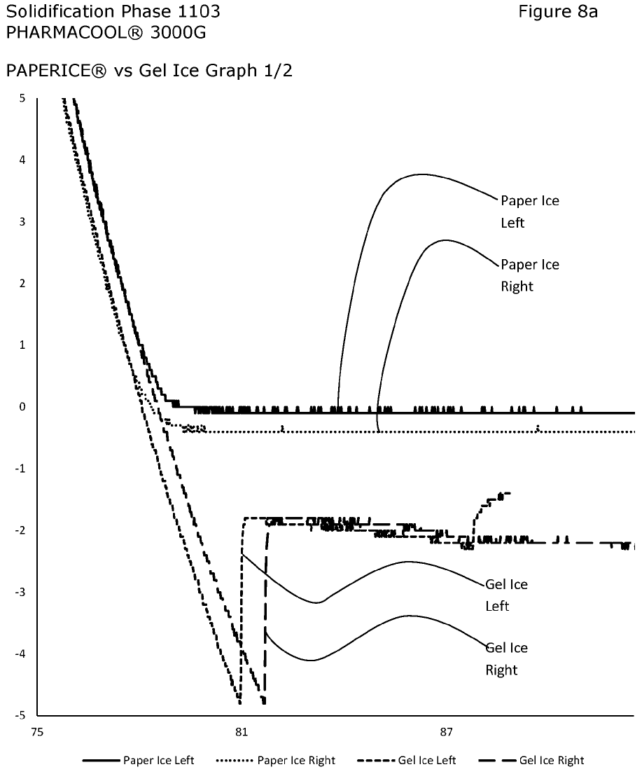

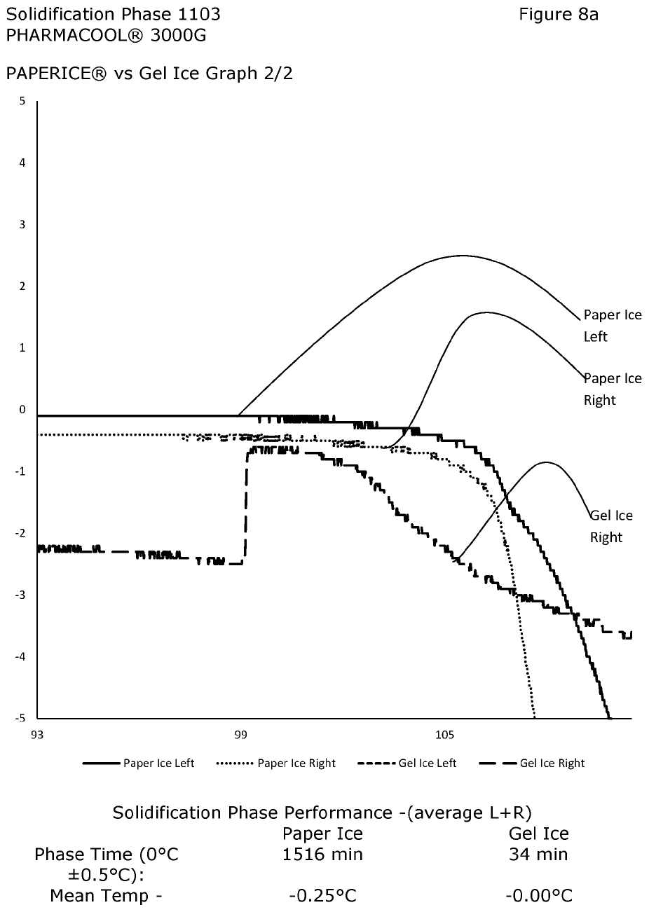

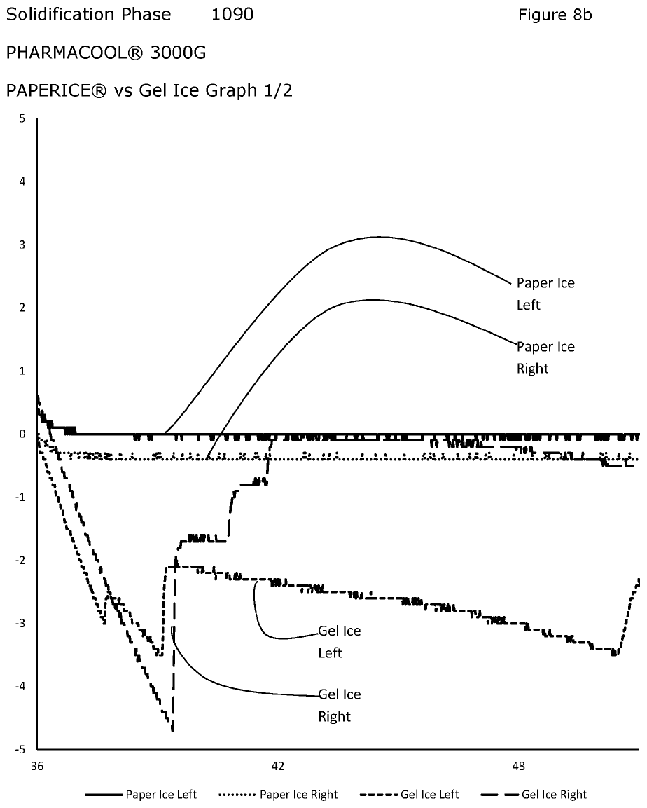

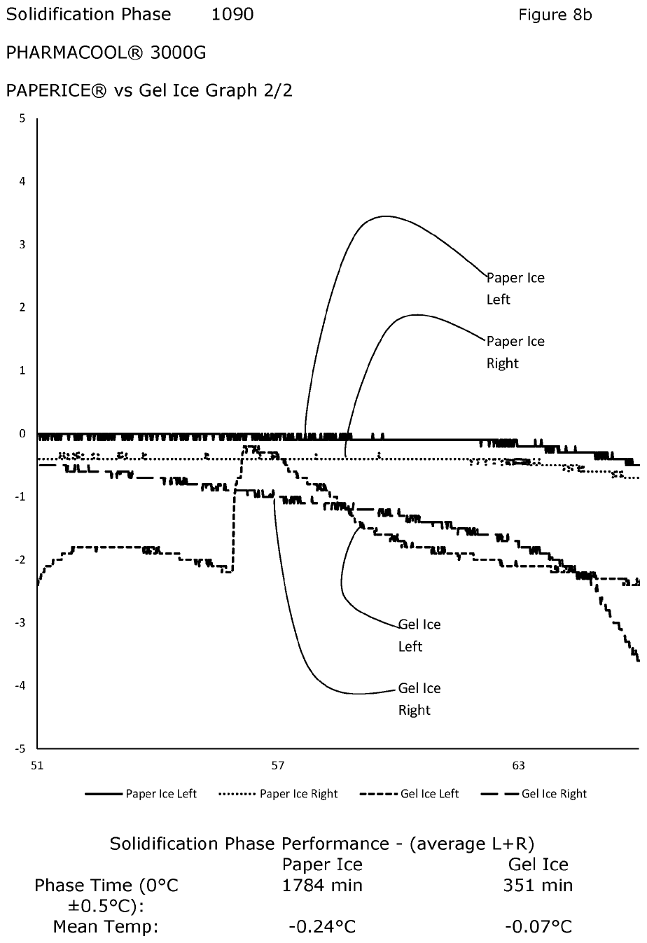

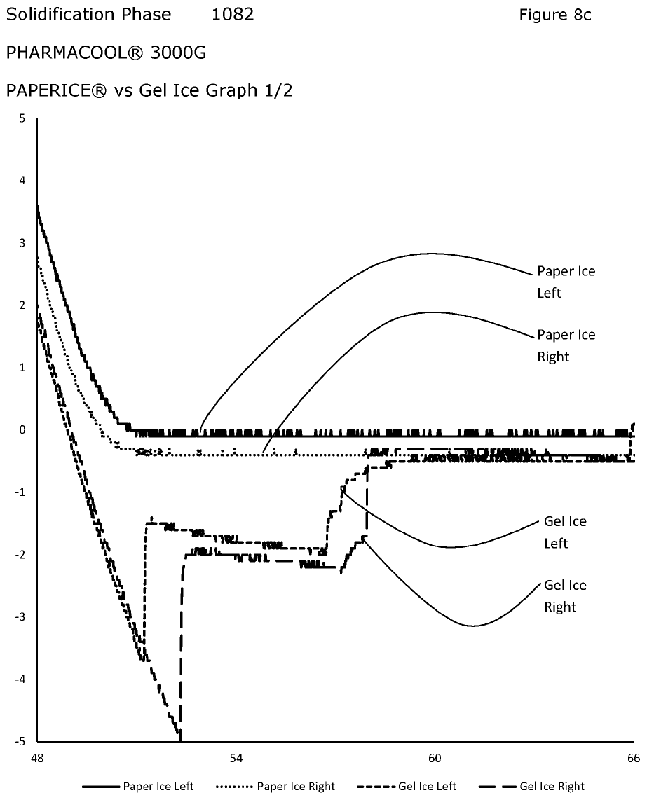

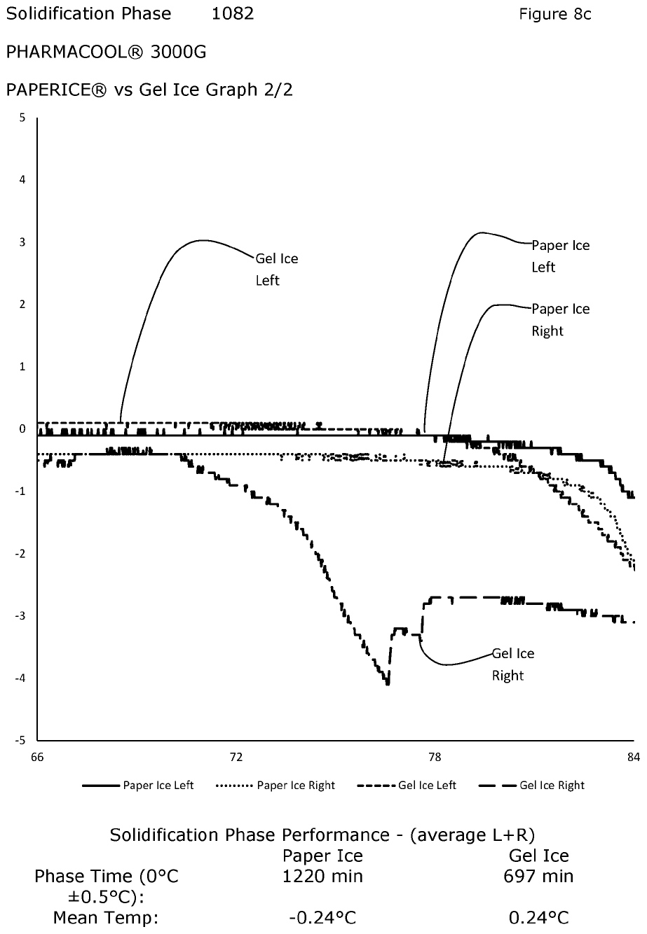

An analysis of the results show that in the solidification phase (i.e. with a decrease of temperature), the prior system gel ice formulations exhibit supercooling characteristics for the gel ice phase control media see the maximum per FIG. 8a being −4.8° C.—as opposed to being within a preferred range of 0.49° C.-0.49° C. This is extremely undesirable given that in the use of phase change systems, medical supply customers and distribution companies, in relation to the transport and storage of such products need to warrant that there has been no deviation from permitted temperature ranges, typically 2-8° C., for winter pack out scenarios where only chilled coolant, at 5.0° C., may be used to protect the system dropping below −0.49° C.—noting that a phase change material, in use, will be placed about a carton, box or other container to shield a product from ambient conditions and will be conditioned at a temperature above the solidification temperature, the phase change material releasing heat from to ensure that a specified temperature range within a payload region of the carton, box or other container is not compromised.

With reference to the first freeze-thaw test results, as shown in FIGS. 7a & 8a, and discussed above a sharp reduction in the prior system temperature—that of supercooling was indicated as an initial event. It is also notable that whilst the temperature of the known gel would appear to have first achieved a temperature of 0° C., the ensuing instability e.g. with regard to FIG. 7a, the temperature per a first probe did not stabilize until some twenty hours after the passing through the 0° C. threshold, whilst it was some ten hours after the passing through the threshold for a second probe when it failed. In contrast, first and second probes with respect to a cellulose fibre phase change packet in accordance with the invention deviated to an insignificant degree after reaching, respectively, temperatures of −0.1° C. and −0.3° C. Equally notable is the fact that period of constant temperature, for each of the probes was 25 hours: compared with the prior gel phase change medium, the first and second temperature probes, provided distinct periods greater than twenty hours in duration, prior to dropping in temperature with regard to the test chamber. It is also notable that the prior phase change medium dropped in temperature at an earlier time than the cellulose fibre phase change medium in accordance with the present invention. Indeed, the averaged results show that the period of solidification time for the present invention was a period of 1516 minutes, with a mean temperature of −0.25° C. whilst the corresponding time and temperature for the prior system was 34 minutes and −0.25° C., respectively, albeit the mean temperature determined arose merely through the temperature passing through the preferred region towards a supercooling event.

With reference to the second freeze-thaw test results, as shown in FIGS. 7b and 8b, a sharp reduction in the prior system was noted to be further distinct: an analysis of the results show that the averaged results show that the period of solidification time for the present invention was a period of 1784 minutes, with a mean temperature of −0.24° C. whilst the corresponding time and temperature for the prior system was 351 minutes and −0.07° C., respectively. It is clear from the traces for the gel ice probes, indications of supercooling were abundant, with temperature excursions to less than −4.5° C., less than −3.3° C. & less than −2.2° C. and obviously would present problems. The third freeze-thaw test results as shown in FIGS. 7c and 8c show that the averaged results show that the period of liquefaction phase time for the present invention was a period of 1220 minutes, with a mean temperature of −0.24° C. whilst the corresponding time and temperature for the prior system was 697 minutes and 0.30° C., respectively. The third freeze-thaw test results also show that the traces for the gel ice probes provided widespread indications of supercooling, with temperature excursions to less than −5.0° C., less than −2.2° C. & less than −4.2° C. and obviously would present problems.

With reference to the first thaw test results, as shown in FIG. 7a, 9a, the prior system was noted to be further distinct: an analysis of the results show that the averaged results show that the period of the liquefaction phase time (from the solid state) for the present invention was a period of 641 minutes, with a mean temperature of −0.24° C. whilst the corresponding time and temperature for the prior system was 623 minutes and 0.16° C., respectively. The second thaw test results, per FIG. 9b, show that the averaged results show that the period of the liquefaction phase time for the present invention was a period of 761 minutes, with a mean temperature of −0.11° C. whilst the corresponding time and temperature for the prior system was 622 minutes and 0.30° C., respectively. The third thaw test results, per FIG. 9c, show that the period of solidification time for the present invention was a period of 715 minutes, with a mean temperature of −0.17° C. whilst the corresponding time and temperature for the prior system was 697 minutes and 0.13° C., respectively.

An analysis of the liquefaction phase (i.e. with an increase of temperature), in a freeze-thaw cycle, the gel ice formulations exhibit similar characteristics with respect to cellulose water formulation in accordance with the present invention for the gel ice phase control media, but this is less important since the issues of concern arise on a reduction of temperature, with supercooling. With regards to the data graphs, it is clear that the present invention provides a simple to make, perform and dispose of ecologically whilst removing any danger of supercooling, which has been known for several years in the industry as providing significant problems in the transport of temperature sensitive goods. It is noted that in the use of phase change systems, medical supply customers and distribution companies, in relation to the transport and storage of such products need to warrant that there has been no deviation from permitted temperature ranges, typically 2-8° C.

With regard to the polymer coated paper data, Applicant Company has performed in-house MVTR tests, carried out in a chamber at 23° C. and 40% Relative humidity, we have set a bench at a loss rate of 5% of the pack weight 1 Kg, noting that a barrier should operate for at least 6 months. Applicant Company has determined that a paper by Mondi—their “Mondi 70/30” paper, with a 30 gsm PE on a 70 gsm paper base was found to provide good results and within the recycling contamination limits.

An increasing level of environmental consciousness has arisen in recent years, supported by legislation from governments around the world, has encouraged industrial researchers to develop the use of eco-friendly, sustainable, and biodegradable materials.

With regard to the wood pulp product, the fibres overlap forming a massive network of tunnels in all directions throughout the pulp product. The chemical molecules making up the fibres are attractive to water molecules. Adhesion is the name of the force of attraction between two unlike molecules. The water molecules and paper molecules are holding on to each other and helping each other move. Once the fibres are covered, the space between the fibres would be empty (technically filled with air), but the water molecules have a strong attraction for each other. Cohesion is the force of attraction between like molecules. Essentially, cohesion and adhesion are the “stickiness” that water molecules have for each other and for other substances. Hydrogen bonding is what makes this possible. Indeed, the water molecules holding onto the fibre walls will also hold onto water molecules and will “bridge” between water molecules that span the space between the fibres. In other words, the gaps between the fibres will with water. As the water molecules move up the fibre elements the water molecules spanning the space of the volume of the pulp product are pulled along. This is called capillary action. Cohesive forces are responsible for surface tension, the tendency of a liquid's surface to resist rupture when placed under tension or stress. Water molecules at the surface (at the water-air interface) will form hydrogen bonds with their neighbours, just like water molecules deeper within the liquid. However, because they are exposed to air on one side, they will have fewer neighbouring water molecules to bond with, and will form stronger bonds with the neighbours they do have. Adhesion is the attraction of molecules of one kind for molecules of a different kind, and it can be quite strong for water, especially with other molecules bearing positive or negative charges. Without being bound by theory, the present invention benefits from having unsaturated fibres permitting a change in phase to be regularized, without giving rise to supercooling in conditions of reducing temperature, which is of benefit in terms of meeting requirements for the maintenance of temperatures in cold chain systems.

The interest in natural plant fibres (fluff, flax, hemp, jute, kenaf, etc.) in industrial usage has grown quickly in the last decade. Several advantages in comparison with synthetic fibres can explain it. They have low density, are annually renewable, and therefore are low in cost. Natural fibres are biodegradable, are crucial at the end of life of products, and have comparable specific strength and modulus as “traditional” glass fibres. Industries such as automotive and construction have started the manufacturing of products using natural fibre, to improve the environmental impact of the product due to the inexpensive price of natural reinforcement.

The timber resources used to make wood pulp are referred to as pulpwood. While in theory, any tree can be used for pulp-making, coniferous trees are preferred because the cellulose fibres in the pulp of these species are longer, and therefore make stronger paper. Some of the most commonly used softwood trees for paper making include spruce, pine, fir, larch and hemlock, and hardwoods such as eucalyptus, aspen and birch. There is also increasing interest in genetically modified tree species (such as GM eucalyptus and GM poplar), because of several major benefits these can provide, such as increased ease of breaking down lignin and increased growth rate. Nonetheless, it is important to confirm that the provision of cellulose fibres is not limited to wood pulp: fibres obtained from non-wood pulp such as bamboo, cotton rags or from linters (short fibres discarded by the textile industry), can also be used in the present invention.

As noted above, it has been found that is has been beneficial to employ a non-saturated sponge, which has benefits in that the resilience of the resultant product in a non-frozen state is beneficial in relation to the absorption of physical shock are improved—i.e. the sponge doesn't become supersaturated and potentially burst the container; moreover, upon freezing, the likelihood of a formation of water ice crystals outside of the sponge is minimized. That is to say, it is believed that the hydrated cellulose fibres can assist in nucleation of ice crystals in a more uniform fashion as the energy in a body of a PCM in accordance with the present invention is reduced, the non-supersaturation of the cellulose fibres ensuring that the body of water is not in a free state as such.

The sponges/foams, being the subject of the present invention can are also referred to as absorbent foams/sponges. The said sponges/foams are biodegradable as the ability of a polymer to be acted upon biochemically in general by living cells or organisms or part of these systems, including hydrolysis, and to degrade and disintegrate into chemical or biochemical products. Further, the invention is bioresorbable, i.e. it comprises an ability of being completely metabolized by the human or animal body making this packing suitable for internal body application. Patent applications are not required to explain how or why something works; the criterion for the invention is: “Whether or not the subject matter is reproducible?”. The answer to that question is found in the examples of the present invention demonstrating the working of the invention and illustrating the required degree of reproducibility.

1. A cold chain system phase change material package, the package comprising a container and a phase change material;
wherein the container includes a PCM absorbent body fabricated from a fibre-based cellulose material; and, wherein the phase change material is principally water.

2. A cold chain system package according to claim 1, wherein the PCM absorbent body is retained within a fibre based sheet material container, the container being formed from and sealed into a tube or tube-like shape, the tube or tube-like shape having an axis, is sealed across the axis to define a closed container.

3. A cold chain system PCM package according to claim 2 wherein the fibre based sheet material is folded to define a rectangular prism.

4. A cold chain system PCM package according to claim 2, wherein the fibre based sheet material comprises wood/cellulose fibre.

5. A cold chain system PCM package according to claim 1, wherein the fibre based material is employed as a loose fill fibre.

6. A cold chain system PCM package according to claim 2, wherein the fibre based sheet material is provided with a plastics waterproof layer.

7. A cold chain system PCM package according to claim 6, wherein the plastics waterproof layer is applied as a hot-melt product.

8. A cold chain system PCM package according to claim 2, wherein the fibre based sheet material is provided with a mineral layer.

9. A cold chain system PCM package according to claim 8, wherein the fibre based sheet material comprises a pliable mineral composite including a thermo-formable bonding agent sufficient to enable a storage article containing the mineral layer to be formed into a selected shape via a thermo-forming process and/or pressure/vacuum forming process.

10. A cold chain system PCM package according to claim 1, further comprising a cellulose pulp insert operable to retain fluid.

11. A cold chain system PCM package according to claim 1, further comprising a cellulose sponge insert operable to retain fluid.

12. A cold chain system PCM package according to claim 10, wherein the insert is compressed prior to use.

13. A cold chain system PCM package according to claim 10, wherein the insert is 40-95% saturated with water.

14. A cold chain system PCM package according to claim 13 wherein the insert is 45-80% saturated with water.

15. A cold chain system PCM package according to claim 10, wherein the insert is pre-formed into a biscuit/waffle shape.

16. A cold chain system PCM package according to claim 10, further comprising a biocide.
